(19) United States
(12) Patent Application Publication
(10) Pub. No.: US 2003/0036056 A1
(43) Pub. Date: Feb. 20, 2003

(54) INHIBITORS AND TARGET MOLECULE CO-LOCALIZATION

(76) Inventors: JOHN J. ROSSI, Rancho Cucamonga, CA (US); GARRY P. LARSON, San Dimas, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 09/465,925

(22) Filed: Dec. 17, 1999

Related U.S. Application Data
(63) Continuation-in-part of application No. 08/922,471, filed on Sep. 3, 1997, now abandoned.
Application No. 08/922,471 is a continuation of application No. 08/522,356, filed on Sep. 13, 1995, now abandoned.
Application No. 08/522,356 is a continuation-in-part of application No. 08/185,827, filed on Jan. 24, 1994, now Pat. No. 5,827,935.

(51) Int. Cl. C12Q 1/68; C07H 21/04; A61K 48/00; C12N 15/85; C12P 19/34

(52) U.S. Cl. 435/6; 435/91.31; 435/455; 514/44; 536/23.1; 536/24.5

(57) ABSTRACT

The invention provides mechanisms for the co-localization in a living cell of a target molecule and of an inhibitor for the target molecule. The invention also provides novel chimeric tRNA^{Lys}-ribozyme molecules that compete effectively with tRNA^{Lys} for HIV-1 reverse transcriptase binding sites. The chimeric human tRNA^{Lys}-ribozymes inhibit HIV reverse transcription by delivering inhibitors such as ribozymes of HIV-1 reverse transcriptase directly to the virion particle and render it non-functional. The chimeric molecules of the invention thus serve as highly specific non-toxic therapeutic agents and vaccines for viral, including lentiviral, infections. These chimeric molecules also reveal a novel, site specific RNA cleaving activity of HIV-1.

### CROSS-REFERENCE TO RELATED APPLICATIONS

This application is a continuation-in-part of application Ser. No. 08/922,471 filed Sep. 3, 1997, which is a continuation of application Ser. No. 08/522,356 filed Sep. 13, 1995, now abandoned; which was a continuation-in-part of application Ser. No. 08/185,827 filed Jan. 24, 1994, now U.S. Pat. No. 5,827,935.

### STATEMENT REGARDING FEDERALLY SPONSORED RESEARCH OR DEVELOPMENT

This invention was made with government support under Grant No. AI 25959 awarded by the National Institutes of Health. The government has certain rights in the invention.

### BACKGROUND OF THE INVENTION

1. Field of the Invention

This invention relates to mechanisms for bringing two or more molecules together in a living cell. More particularly, the invention relates to mechanisms for bringing together within a cell a target molecule and an inhibitor so as to increase the concentration of the inhibitor with respect to the target. For example, the invention relates to mechanisms for increasing the cellular concentration of a ribozyme with respect to a target mRNA molecule to be cleaved by the ribozyme.

One embodiment of this invention relates to chimeric tRNA^{Lys}-ribozyme constructs which compete effectively with tRNA^{Lys} for binding to HIV-1 reverse transcriptase. These chimeric molecules provide a co-localization mechanism for delivering inhibitors of HIV-1 and reverse transcriptase to the virion particle itself.

2. Description of the Related Art

RNA is unusual in its ability both to store information in its nucleotide sequence and to function as an enzymatic catalyst of specific reactions (1, 2). This combination of attributes has created opportunities for engineering RNA enzymes (ribozymes) which can be used to cleave and functionally inactivate targeted RNAs. Some of the attributes of ribozymes which make them attractive candidates for therapeutic agents are their ability to site-specifically cleave targeted RNAs, cleave multiple substrates, and their ability to be engineered for improved cleavage specificity and enhanced catalytic turnover (3, 4). There are five catalytic motifs which have been successfully modified and/or adapted for use in ribozyme applications. These are the group I introns, RNAse P, the hammerhead and hairpin motifs, and the self-cleaving domain of the hepatitis delta virus (5, 3, 6, 7). Each of these engineered ribozymes only requires a divalent metal cation for activity (usually Mg⁺⁺) which participates in the chemistry of the cleavage reaction (8, 9, 10).

The therapeutic use of ribozymes is an attractive goal which merges the basic and applied sciences. Since all genes are expressed through RNA intermediates, the potential applications are limited primarily by knowledge of the disease or disease associated with a given RNA. In the case of viral infections, such as HIV, ribozymes can be tailor made to cleave viral transcripts, thereby leaving cellular transcripts untouched. Because of this, HIV is a prime target for ribozyme inactivation. This concept was successfully tested by intracellularly expressing a hammerhead ribozyme targeted to a gag cleavage site, which resulted in up to a 40 fold reduction in viral p24 antigen production in HeLa CD4⁺ cells challenged with HIV (11, 12). Because a retrovirus has an RNA genome, there are hundreds of potential ribozyme cleavage sites along the length of the viral genomic and subgenomic RNAs. Since the virus mutates rapidly, and can become resistant to most drugs developed to inhibit a single viral target (13), ribozymes have become an important alternative for anti-viral therapeutic agents since multiple ribozymes targeted to a number of different sites can be simultaneously delivered to cells for inhibition of HIV (14). There are two times in the viral life-cycle when ribozymes could be effective against HIV infection. The first immediately following infection prior to proviral DNA formation, when all or part of the viral genome is still in the form of RNA, and the second following the establishment of integrated provirus from which spliced and a full length viral transcripts are produced (15, 16). An important consideration is the observation that HIV can infect quiescent T-lymphocytes, wherein proviral DNA synthesis is initiated but is incompletely reverse transcribed (17). If a ribozyme is present in the infected cell cytoplasm, it theoretically could protect cells from permanent infection by cleaving the RNA at this early step, before the T-cell becomes activated. In support of this type of action by a ribozyme, Yamada, et al., (18) have demonstrated a 50-100 fold reduction in HIV proviral DNA formation in cells expressing a hairpin ribozyme targeted to a site in the 5′ leader sequence.

A number of reports demonstrating varying levels of ribozyme mediated protection of cultured cells from HIV infection have been published (14, 19, 20, 21, 22, 12, 23, 24). The most encouraging results of ribozyme mediated inhibition of HIV utilized a hairpin ribozyme targeted to a highly conserved GUC cleavage site in HIV (22, 18, 24). Expression of this ribozyme gave rise to long term resistance to infection, including resistance to a variety of HIV isolates. Studies recently completed demonstrate that hammerhead ribozymes targeted to conserved sites in the tat and a shared tat-rev exon, when expressed from a Moloney viral vector long terminal repeat (LTR) can confer protection to cells in culture for at least 21 days (50, incorporated herein by reference). Despite these reported successes, the observation has been made that ribozyme mediated protection of cells can be overcome with increasing multiplicity of infection, and in some instances with prolonged culture times. In a patient setting, this is likely to be a serious problem since there is substantial evidence suggesting that the virus is highly concentrated in the lymphoid system, providing, in essence, a high multiplicity of infection to CD4⁺ cells entering that environment (25, 26, 27). A somewhat different problem is that of the genetic variability of HIV (15). Although it has not been formally demonstrated in experimental models, it is reasonable to assume that viral resistance to ribozymes can and will occur, especially in a patient setting where the pool of viruses is bound to be genetically heterogeneous (28, 13, 29, 30).

The first steps in solving this problem involve developing a detailed understanding of how ribozymes can be made to function more effectively in an intracellular environment. For most RNAs, very little is known about the mechanisms regulating the pathway of movement from transcription through translation and, in the case of HIV, from transcription through packaging. There is increasing evidence, although some of it still controversial, that nuclear transcripts are processed and migrate along specific tracks which predicts non-uniform distributions of specific nuclear transcripts (31). Following export from the nucleus, there is also increasing evidence that a variety of RNAs can be specifically localized within the cytoplasm as well (32).

From the prospective of ribozyme therapeutic applications, capitalizing upon the localization properties of RNAs could facilitate intracellular functioning of ribozymes by allowing them to co-localize with their target RNAs. Sullenger and Cech (1993) (33) (incorporated herein by reference) have directly tested this idea by utilizing the dimerization and packaging signal of a Moloney murine leukemia virus genomic RNA to co-localize a hammerhead ribozyme with its target, the lacZ gene carried by another recombinant Moloney viral vector. They found that up to 90% inhibition of infective virus production could be achieved as a result of co-packaging the ribozyme and the lacZ target containing viral RNAs. Their data showed that inhibition of lacZ expression was only achieved when the ribozyme was co-packaged with the genomic target RNA. Thus, mRNAs harboring the lacZ sequence, but lacking the packaging signal, were not destroyed by the ribozyme, clearly demonstrating the usefulness of a co-localization strategy for ribozymes.

A different co-packaging strategy which takes advantage of the fact that HIV utilizes tRNA₃^{Lys} as a site specific primer for reverse transcription is described in U.S. Pat. No. 5,827,935. A tRNA₃^{Lys}-ribozyme construct is utilized to co-localize the ribozyme to the HIV-1 sequences immediately 5′ or upstream of the primer binding site of the tRNA₃^{Lys}. The strategy in that application is that the chimeric tRNA₃^{Lys}-ribozyme constructs could be bound by HIV reverse transcriptase and encapsulated during viral assembly, thereby rendering those viral particles non-functional.

During the series of events that RNAs undergo from their birth to their death, they are constantly associated with proteins (34). It is a virtual certainty that ribozymes will encounter proteins in an intracellular environment which will have an effect, either positive or negative, on their activity. Tsuchihashi, et al. (1993) (35), Herschlag, et al., (1994) (36) and Bertrand and Rossi (1994) (37) have observed RNA binding proteins such as HIV-1 encoded NCp7 and cellular hnRNP A1 can facilitate ribozyme catalytic turnover in vitro.

The specificity of the interactions of HIV reverse transcriptase (RT) with cellular tRNA₃^{Lys}, the primer tRNA used by all the mammalian lentiviruses, is well established. It has been demonstrated that the entire tRNA^{Lys} molecule as well as various segments of the tRNA per se are capable of interacting specifically with HIV-1 RT (51, 52, 53). This tRNA is selectively bound by RT and in the presence of the nucleocapsid protein NCp15 (or NCp7), unwinds the aminoacyl stem of the tRNA, allowing it to base pair with the viral primer binding site (PBS) (38). The premise of the '935 patent is that a ribozyme appended to the 3′ terminus of tRNA₃^{Lys} could be captured by RT, co-packaged with the virus, and the ribozyme would be aligned to cleave the viral genomic RNA and destroy its infectivity. Available data supports the hypothesis. These data are summarized as follows: (1) The tRNA-ribozyme binds selectively to HIV RT with a binding affinity virtually identical to a synthetic tRNA₃^{Lys}. (2) The tRNA-ribozyme construct is expressed as a Pol III transcript when transfected into 293 cells, and the ribozyme moiety is not processed from the transcript, although the 5′ precursor segment of the tRNA-ribozyme is processed normally. By including the CCA (which is normally added post transcriptionally to the tRNA), along with a one base deletion in the T stem, these molecules are not subject to the normal 3′ processing events. (3) The tRNA-ribozyme is exported to the cytoplasm, making it available for binding with RT. (4) When the tRNA-ribozyme is transiently transfected into 293 cells, there are equivalent levels of tRNA-ribozyme transcript to endogenous tRNA₃^{Lys}. (5) Co-transfection of the tRNA-ribozyme gene with pNL4-3 DNA into 293 cells resulted in a 4 to 12 fold reduction in infectious virus production relative to control constructs. See FIG. 5.

Prior to this invention there has been no report of chimeric tRNA^{Lys}-ribozyme molecules.

### DEFINITION

Co-localization: As used in this application, the term co-localization means the positioning of two or more molecules within a living cell, one of which is a target and the other an inhibitor of the target such that the concentration of the inhibitor with respect to the target is increased within the cell and function or expression of the target is constrained or inhibited.

Co-localization may be accomplished by covalent linkage (cis-ribozyme) or via co-targeting the viral capsid. A specific embodiment of co-localization pursuant to this invention entails the positioning within a living mammalian cell of a ribozyme adjacent a virion particle to cleave virion RNA.

### SUMMARY OF THE INVENTION

This invention provides co-localization mechanisms and living cells in which an inhibitor and a target are co-localized by such mechanisms. An important object of the invention is to provide novel intracellular immunogens for vaccines against viral infections.

One preferred embodiment of this invention provides novel chimeric tRNA^{Lys}-ribozyme molecules that compete effectively with tRNA^{Lys} for HIV-1 reverse transcriptase binding sites. The chimeric human tRNA^{Lys}-ribozyme inhibit HIV reverse transcription by delivering inhibitors such as ribozymes of HIV-1 RT directly to the virion particle and render it non-functional. The chimeric molecules of this invention thus serve as highly specific non-toxic therapeutic agents.

These chimeric molecules also reveal a novel, site specific RNA cleaving activity of HIV-1.

### DESCRIPTION OF THE FIGURES

FIG. 1. (SEQ ID NO. 1 and SEQ ID NO. 2) shows the structure of one chimeric tRNA^{Lys}-ribozyme construct. This construct has been cloned into a BLUESCRIPT™ (Stratagene, La Jolla, Calif.) transcription vector using Sacll and Xhol restriction sites. Following linearization at the Sacll site the chimeric RNA can be transcribed in vitro using bacteriophage T7 RNA polymerase. The tRNA^{Lys}-ribozyme construct is shown base-paired to the PBS of HIV-1 genomic RNA, and the ribozyme flanking sequences are shown base-paired to the AUC cleavage site indicated on the HIV genomic RNA strand. The ribozyme moiety is followed by five uracil residues, which act as an RNA polymerase III transcription terminator. The G*A mismatch shown at the 3′ end of the ribozyme/HIV RNA duplex was included to eliminate premature transcription termination, as the perfectly matched ribozyme pairing arm would have a stretch of four uridines. There is a MaeI restriction site in between the tRNA and ribozyme moieties, allowing the tRNA to be transcribed independently of the ribozyme.

FIG. 2. This gel shift experiment shows binding of the chimeric tRNA^{Lys}-ribozyme to HIV-1 reverse transcriptase. The eight lanes of the gel from left to right are:

1. Molecular weight marker.

2. tRNA^{Lys} in vitro transcript which has extra bases at both the 5′ and 3′ ends. The extra 5′ bases are from the BLUESCRIPT™ poly linker between the T7 promoter and the Xhol site. There are six extra nucleotides at the 3′ end derived from the nucleotides after the CCA of the tRNA to the MaeI site which separates the tRNA from the ribozyme.

3. tRNA^{Lys}-ribozyme in vitro transcript which has the same extra 5′ bases as tRNA^{Lys}, but terminates at Sacll site at the end of the ribozyme moiety.

4. tRNA^{Lys} transcript incubated with HIV-1 reverse transcriptase.

5. tRNA^{Lys}-ribozyme transcript incubated with HIV-1 reverse transcriptase.

6. tRNA^{Lys} transcript incubated with avian myeloblatosis virus (AMV) reverse transcriptase.

7. tRNA^{Lys}-ribozyme incubated with AMV reverse transcriptase.

8. tRNA^{Lys} with competing, non-radioactively labeled tRNA^{Lys}-ribozyme incubated with HIV-1 reverse transcriptase.

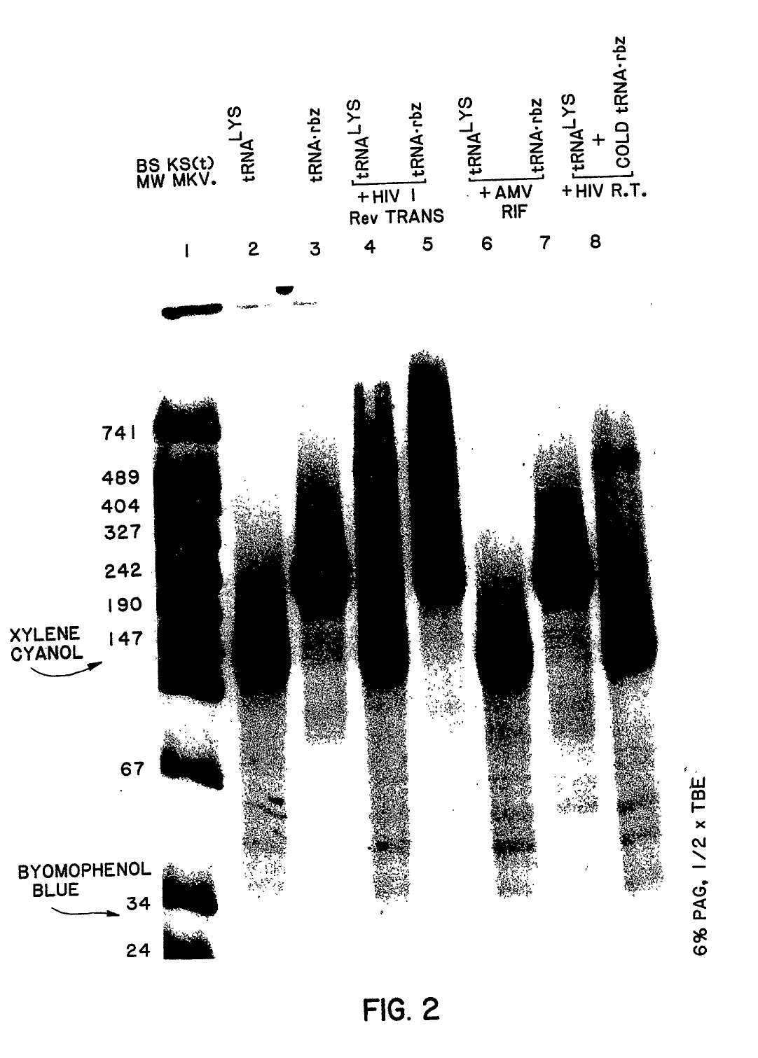

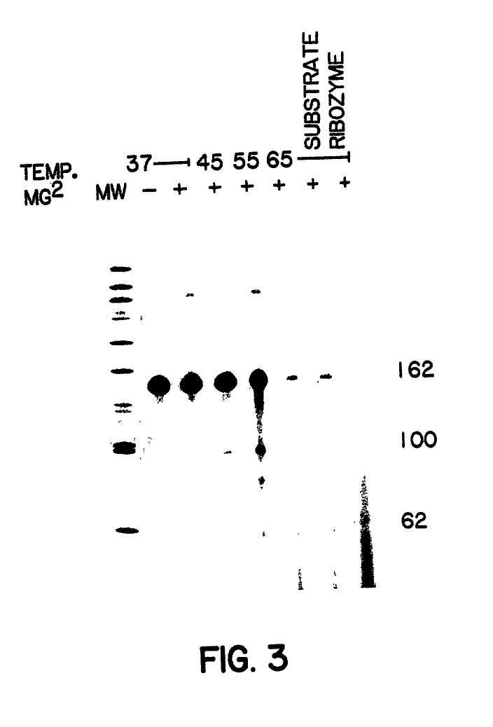

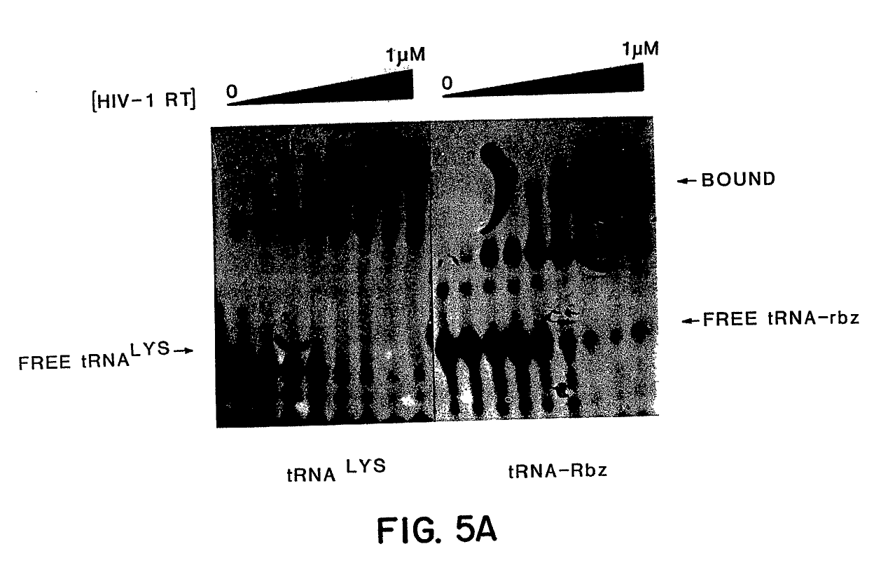

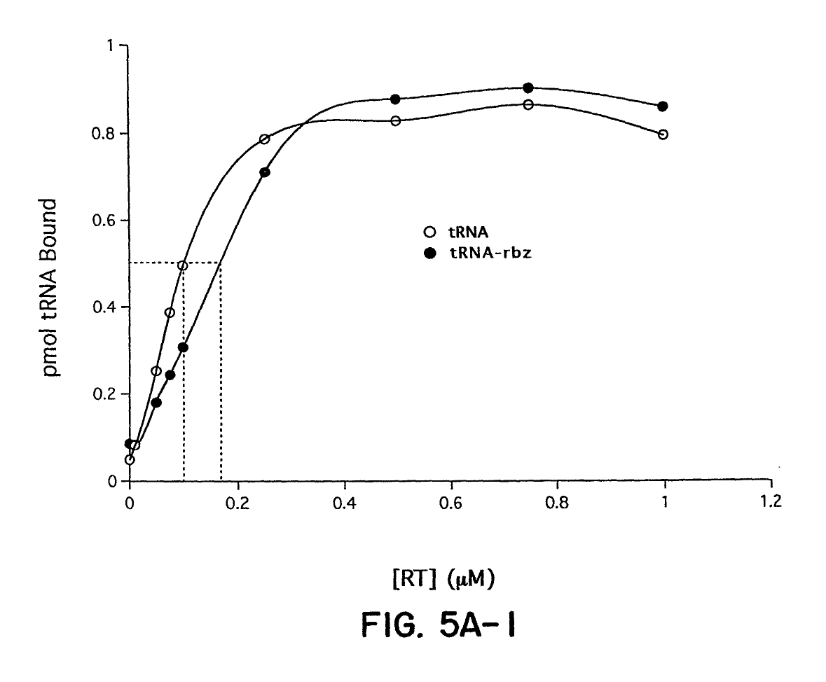

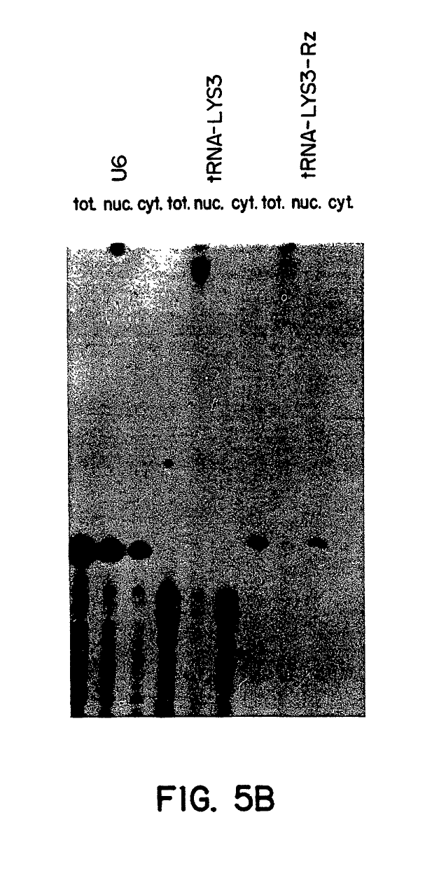

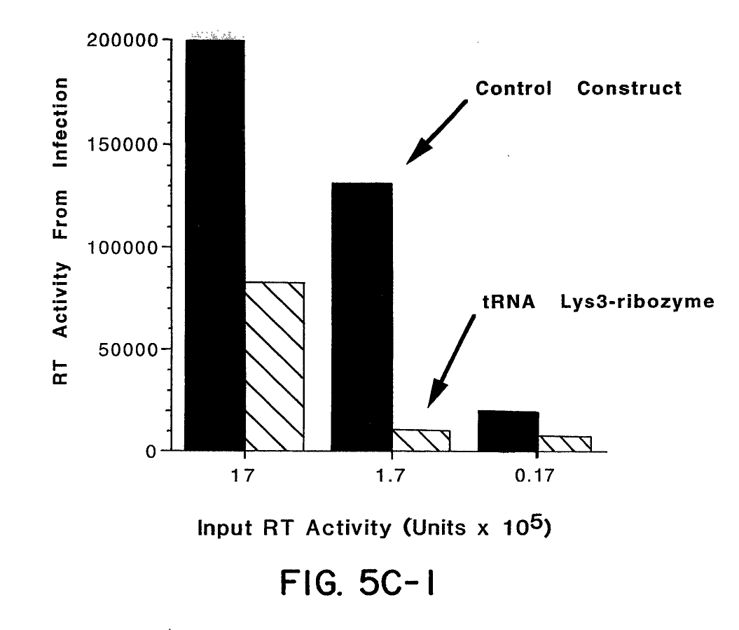

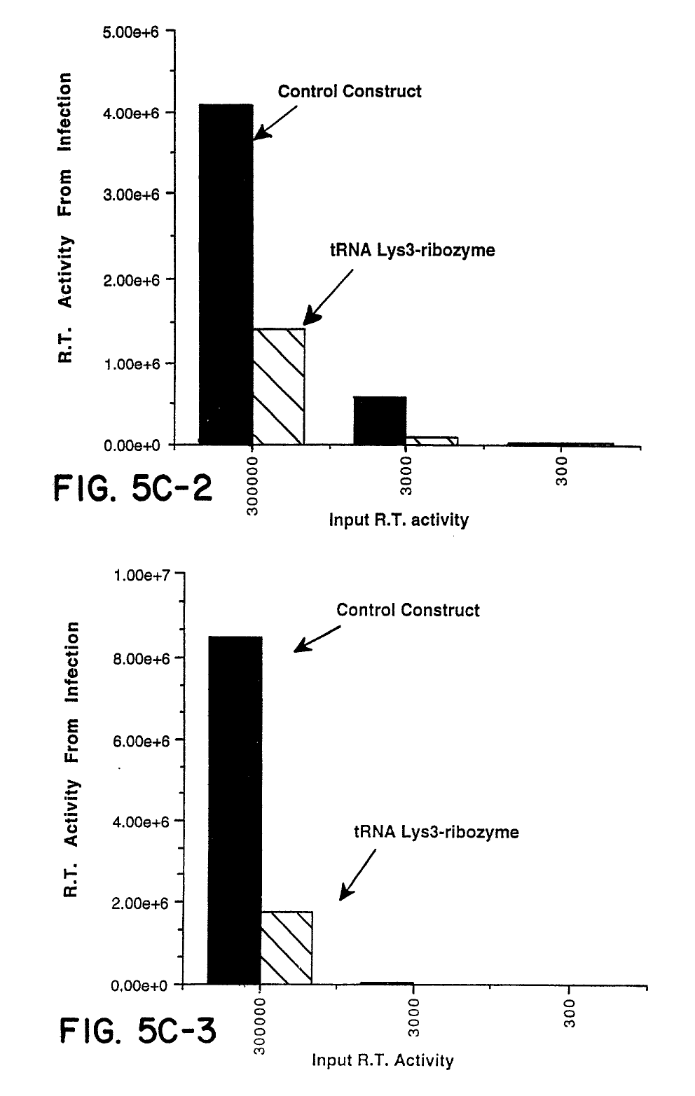

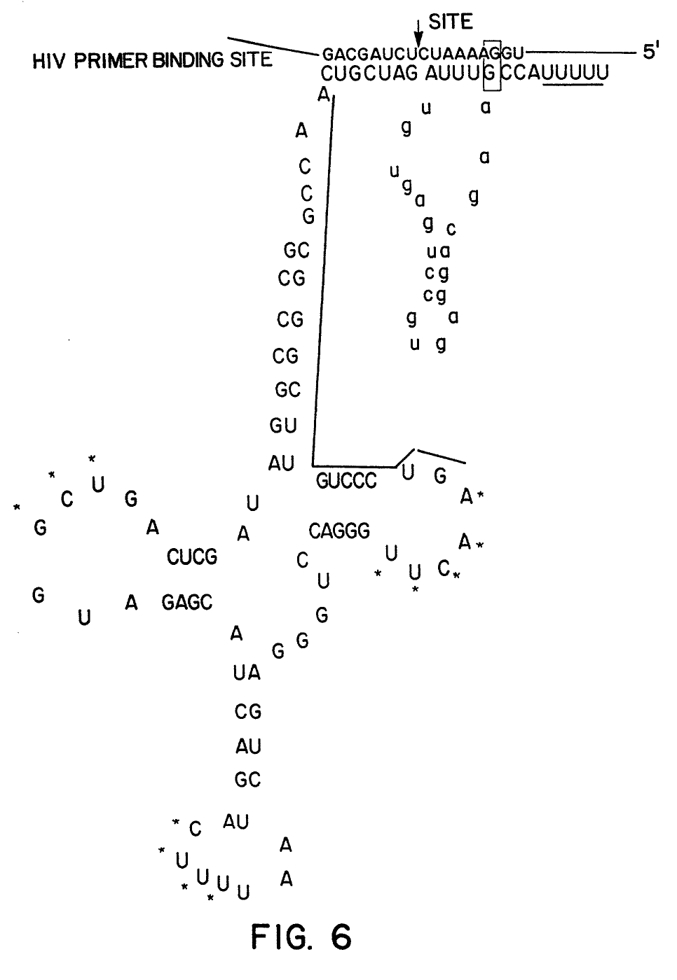

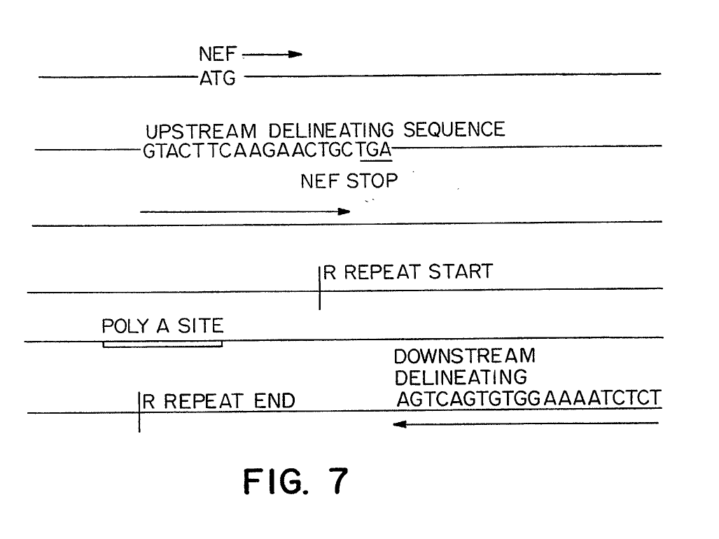

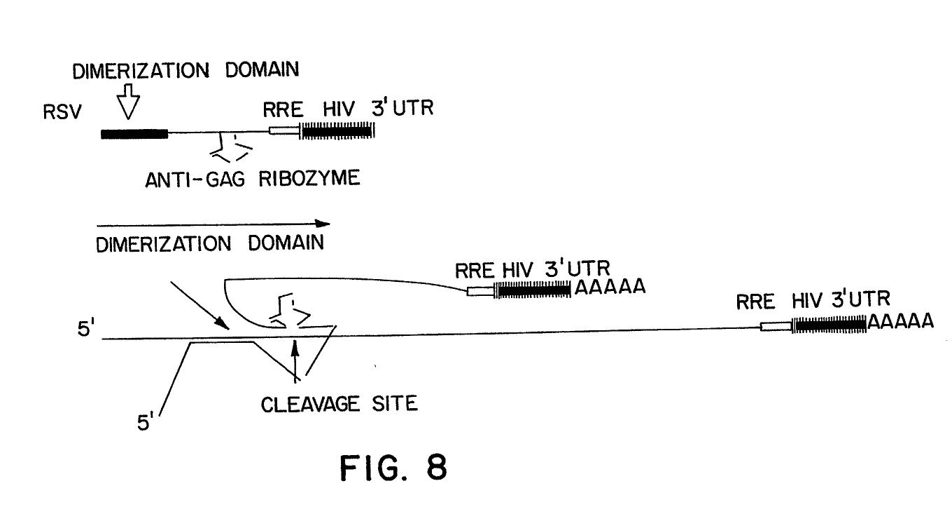

FIG. 2 shows that the chimeric tRNA^{Lys}-ribozyme specifically binds to HIV-1 RT by a shift in radioactivity when HIV-1 RT is present. Cold tRNA^{Lys}-ribozyme competes with tRNA^{Lys} for binding to HIV-1 RT as indicated by the reduced radioactive shift in lane 8.

FIG. 3. This experiment demonstrates cleavage of a 162 nucleotide, radioactively labeled HIV-1 RNA containing the primer binding site plus sequences upstream of this and including the AUC cleavage signal for the ribozyme. The cleavage products are 101 and 61 bases. The extent of cleavage increases with increasing temperature.

FIG. 4. [Intentionally skipped]

FIG. 5A. Illustrates HIV-1 RT binding to tRNA₃^{Lys} and tRNA₃^{Lys}-ribozyme.

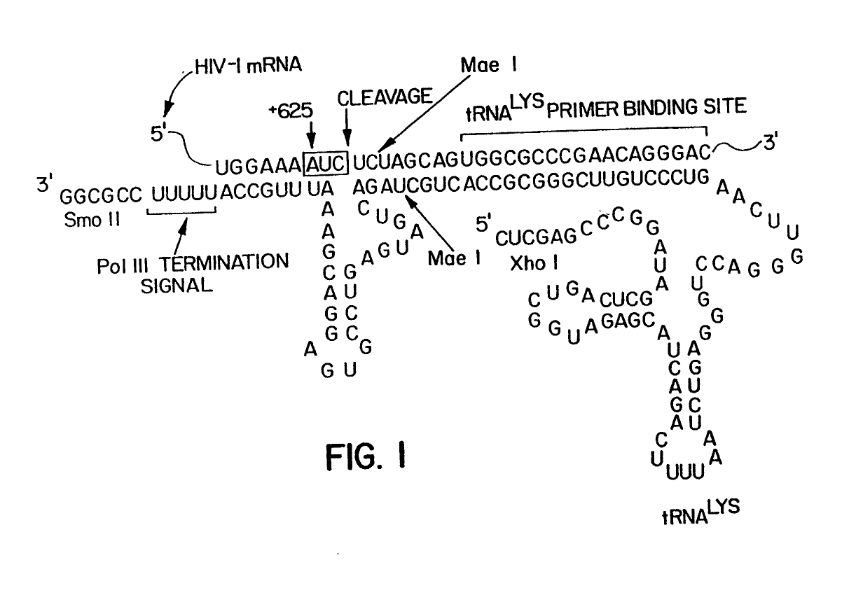

FIGS. 5A-1. This graph demonstrates that tRNA₃^{Lys} and tRNA₃^{Lys}-ribozyme have similar binding affinity for HIV-1 RT.

FIG. 5B. Primer extension analyses demonstrating nuclear localization of chimeric transcript. The primer for the tRNA₃^{Lys}-ribozyme is in the ribozyme moiety, and the primer for tRNA₃^{Lys} is at the 3′ end of the tRNA. This illustrates the presence of the entire transcript.

FIG. 5C. Results of infectious virus assays carried out with supernatents from 293 cells transfected with tRNA-ribozyme or control construct (ribozyme minus tRNA in same vector) and co-transfected with pNL4-3. Three independent experiments are presented.

FIG. 6. Illustrates the tRNA^{Lys}-ribozyme which is the starting molecule. The asterisks indicate sites which UV cross-link to HIV RT or are protected from RNAse digestion in the presence of RT. A deliberately created mismatch in the ribozyme pairing arm is indicated with a boxed in nucleotide pair. This was done to eliminate a stretch of four thymines in the ribozyme gene which could serve as a Pol III termination site. The authentic termination site (5 U's or T's in DAN) is underlined. The T loopstem and aminoacyl acceptor stem which pair with the HIV primer binding site are overlain with a heavy line.

FIG. 7. A schematic representation of nef and 3′UTR region to be included in ribozyme and GH reporter systems. The delineating sequences are the extremities of the DNA amplified by PCR. These sequences are from the pNL4-3 proviral clone and encompass the region of nucleotides 9389 through 9704.

FIG. 8. A construct containing anti-HIV-1 ribozyme expressed in context of dimerization domain and RRE to facilitate co-localization with HIV full-length genomic RNAs.

### DETAILED DESCRIPTION OF THE INVENTION

The invention provides various co-localization mechanisms. These mechanisms include, among others, (i) utilization of specific RNA trafficking pathways to both the target and the inhibitor, (ii) utilization of protein interaction with inhibitor and target molecules, e.g. HIV-1 RT (see Sullenger and Cech (33)), (iii) use of cellular proteins which subcellularly compartmentalize the inhibitor to the target or a specific target site; (iv) use of cis-acting sequence substituents on ribozyme transcripts to direct the ribozyme to a specific subcellular trafficking pattern or site; (v) ribozymes which include any molecule or moiety that specifies a distinct intracellular trafficking pattern and target localization site.

a. tRNA₃^{Lys}-Ribozyme Chimeric Molecules

One of the most important problems facing the routine use of ribozymes as therapeutic agents is that of maximizing effective interactions of ribozymes and target RNAs. It has been convincingly demonstrated by Sullenger and Cech (33) that co-localization of a ribozyme and target RNA through a retroviral packaging signal can dramatically enhance the effectiveness of the ribozyme pairing with, and cleaving its substrate. As noted, the '935 patent describes somewhat different co-localization strategy with the tRNA₃^{Lys}-ribozyme chimeras, which are bound by HIV RT allowing alignment of the ribozyme during packaging of the virus.

This approach has led to a reduction of infective viral titer as a consequence of co-expressing chimeric tRNA-ribozymes with HIV proviral DNA. In order to make this a more generally useful strategy, it is useful to develop chimeric molecules which effectively compete against cellular tRNAs for binding to RT, yet do not create a general toxicity problem. Genetic variants of tRNA₃^{Lys} which maintain the sequence and structural features required for interaction with a ribozyme for cleavage are dissimilar enough from cellular tRNA₃^{Lys} so as not to interfere with normal cellular metabolism. The use of these variants will also be coupled with enhanced intracellular expression systems. The identification of molecules which can still interact with the PBS of HIV (which means leaving at least the 3′ segment of the amino-acyl acceptor stem intact), thereby allowing alignment of a ribozyme (appended to the 3′ end) with a cleavage site adjacent to the viral PBS is contemplated.

b. Increasing Intracellular Levels of tRNA₃^{Lys}-ribozyme

Since high levels of expression of the tRNA₃^{Lys}-ribozyme chimeric gene during transient transfection were observed, it is reasonable that inserting multiple, tandem copies of the tRNA ribozyme chimeric genes in a vector such as adeno associated virus (AAV) can also lead to high level expression.

A potential strategy for increasing the intracellular levels of the chimeric ribozyme transcript is to express them from heterologous promoters. For those variants which lack the A or B boxes, this will be a necessity. For variants which have maintained these elements, site directed changes which eliminate the promoter function will allow testing of these constructs using heterologous promoters. Several candidate promoters have been developed for ribozyme expression for example the human U6 snRNA promotor.

### EXAMPLE 1

The upstream promoter element of the human U6 snRNA gene can be used to drive transcription of the tRNA₃^{Lys}-rbz and increase the levels of expression (55). The human U6 snRNA gene has a Pol III promoter element which is 5′ of the coding sequence (39). Transcription terminates after a string of five uracil residues, resulting in a RNA with well defined ends. It has been demonstrated that this promoter can be used to transcribe ribozyme containing RNAs which localize to the cytoplasm. A potential advantage of this promoter is that transcription can be engineered to initiate at the +1 sequence of the tRNA molecule, thus eliminating any need for processing a 5′ leader, and allowing the synthesis of a very defined transcript.

To compare levels of expression with genes driven by the U6 snRNA promoter, human embryonic kidney cells (293 cells) were transiently transfected with 10 μg of ptRNA₃^{Lys}-rbz, ptRNA₃^{Lys}-mrbz, or pU6tRNA₃^{Lys}-rbz using the CellPhect Transfection Kit (Pharmacia). PTKGH (growth hormone plasmid) (1 μg) was cotransfected with the samples for quantitation of transfection efficiency which was measured at 20%. After 2 days, RNA was prepared from transfected cells by the RNA STAT-60™ reagent (Tel-Test “B”, Inc., Friendwood, Tex.). Total RNA (25 μg) was resolved on 1% formaldehyde-agarose gels, blotted, and hybridized first to a probe specific for the ribozyme moiety, then washed and hybridized to a U6 snRNA probe as a loading control. The oligonucleotide probes were kinased in 10 μl of 70 mM Tris, pH 7.6, 10 mM MgCl₂, 5 mM DTT, 150 uCi [γ-³²P]ATP, 10 U T4 polynucleotide kinase (New England Biolabs), at 37° C. for 30 minutes. Blots were exposed to autoradiograph film overnight. The tRNA₃^{Lys}-ribozyme chimeric transcript was poorly expressed from the tRNA₃^{Lys} intragenic RNA polymerase III promoter in vivo, but use of the human U6 snRNA promoter resulted in a 10-fold to 20-fold increase in expression of the tRNA₃^{Lys}-ribozyme transcript. This difference in transcription levels indicates that the presence of a fully functional internal tRNA promoter does not interfere with transcription by U6 promoter elements.

c. tRNA₃^{Lys}-ribozyme Binding to HIV Reverse Transcriptase to Effect Co-localization

Co-localization of a chimeric tRNA₃^{Lys}-ribozyme with target mRNA is imparted by specific binding of HIV RT to the tRNA₃^{Lys} moiety of the chimeric RNA. Genetic fusions consisting of the entire mature coding sequence or 18 bases of the 3′ end of human tRNA^{Lys} were fused to hammerhead ribozyme containing RNAs with base pairing capabilities to the HIV-1 sequences immediately 5′ or upstream of the PBS. The 3′ terminal 18 nucleotides of the tRNA^{Lys} are complementary to the PBS. These chimeric molecules have been tested in cell free assays for their ability to bind to HIV-1 RT and their inhibitory activity on HIV-1 RT polymerization activity. The ribozyme moiety targets the cleavage of HIV-1 viral RNA at a known hammerhead cleavage site immediately upstream of the PBS for initiation of reverse transcription in the HIV-1 viral RNA. The site chosen for initial study, and reported here is an AUC in which cleavage is immediately after the C. This site is absolutely conserved in all HIV-1 isolates sequenced to date. The chimeric RNAs, which are specifically bound by HIV-1 RT, should be carried into newly formed HIV-1 virions during viral assembly. The chimeric primers effectively block HIV-1 reverse transcription, making them a novel, highly target specific, and unique anti-HIV-l therapeutic agent. In addition, the tRNA^{Lys} portion contains within its mature coding sequence the elements required for transcription by human RNA polymerase III, thereby making it feasible to insert the gene, rather than the RNA, into human cells.

Studies of the binding of the chimeric molecules to HIV-1 RT revealed that the complex of chimeric tRNA^{Lys}-ribozyme, or 18 3′ nucleotides of tRNA^{Lys}-ribozyme, or tRNA^{Lys} with an extra 6 nucleotides appended to the 3′ end, when base paired to the PBS signal of HIV-1 RNA, serves as a substrate for a novel ribonuclease activity associated with HIV-1 RT. This activity results in cleavage of the primer at a site very close to the 3′ end of the tRNA^{Lys} molecule, CCA-3′. This activity is of unknown function in the viral replication cycle, but may play an important role in the use of chimeric RNAs by freeing the ribozyme moiety from the tRNA moiety such that it can cleave one or both of the viral RNAs encapsidated in the HIV-1 virion.

### EXAMPLE 2

To examine the effect that the ribozyme moiety bound to the tRNA₃^{Lys} would have on the interaction of tRNA₃^{Lys} with HIV-1 RT, binding assays were performed at 25° C. for 30 minutes in 10 μl reactions containing 100 nM radiolabeled tRNA₃^{Lys} or tRNA₃^{Lys}-rbz transcripts, 40 mM Tris, pH 8.3, 60 mM NaCl, 6 mM MgCl₂, 5 mM dithiothreitol and 100 nM AMV, MLV, or HIV RT (55). Samples were electrophoresed under nondenaturing conditions at 4° C. in a 4% polyacrylamide gel with 0.5×TBE buffer. An autoradiograph of the polyacrylamide gel electrophoresis of HIV RT complexed with tRNA₃^{Lys} or tRNA₃^{Lys}-rbz reflects a shift in electrophoretic mobility on binding by HIV RT. Both tRNA₃^{Lys} and tRNA₃^{Lys}-rbz interact with HIV-1 RT, whereas they do not interact with AMV or MLV RT under identical conditions. These data demonstrate that both tRNAs share a similar secondary and tertiary structure that allows for the preferential recognition by HIV-1 RT. To demonstrate competition between tRNA₃^{Lys} and tRNA₃^{Lys}-rbz for binding to HIV-1 RT, 4-20 μM nonradiolabeled tRNA₃^{Lys} or tRNA₃^{Lys}-rbz was added after preincubation of HIV RT with 1 pmol radiolabeled tRNA₃^{Lys}-rbz or tRNA₃^{Lys}, respectively. Nonradiolabeled tRNA₃^{Lys}-rbz competes effectively and specifically with radiolabeled tRNA₃^{Lys} for binding to HIV RT, and vice versa.

Native polyacrylamide gel electrophoresis was used to compare the binding affinities of HIV RT to tRNA₃^{Lys} and tRNA₃^{Lys}-rbz by titrating a fixed amount of each RNA with increasing amounts of HIV-1 RT. The tRNA₃^{Lys}-rbz binds to HIV RT width a similar dissociation constant as that of tRNA₃^{Lys}. See FIG. 5A.

### EXAMPLE 3

Based on the recognition of the tRNA₃^{Lys}-rbz by HIV RT in vitro and its efficient expression in transfected cells, it is expected that tRNA₃^{Lys}-rbz transcripts would be incorporated into viral particles (55). These particles would be defective, as the ribozyme moiety would cleave the genome and prevent the initiation of reverse transcription before or soon after entry into a cell.

To test the ability of the tRNA₃^{Lys}-rbz to reduce viral infectivity, we assayed the infectivity of viral particles produced from cells expressing the chimeric constructs. Human 293 cells were cotransfected with proviral HIV-1 DNA and plasmids containing either tRNA₃^{Lys}-rbz or tRNA^{val}-rbz at a ratio of 1:10 HIV/ribozyme using the CellPhect Kit. Supernatants were collected 3 days after transfection, and the RT activity of the supernatant was determined. Viral infectivity was assayed to determine whether the tRNA-rbz rendered the virions defective for subsequent infection. The different supernatants were standardized according to their RT activity and used to infect the T cell line CEMX174. From 3 to 10 days after infection, supernatants were collected and assayed for HIV RT activity and HIV p24 antigen production. HIV RT levels correlated well with p24 antigen results. When p24 antigen production from cells infected with virions produced from the U6tRNA₃^{Lys}-rbz transfected cells was compared with the p24 antigen production from cells transfected with virions produced from cells transfected with U6 vector alone, a 5-fold to 6-fold reduction could be measured, indicating that the ability of the virus to infect had been significantly reduced. Inclusion of the tRNA₃^{Lys} moiety resulted in up to 200-fold reduction in infectivity over the control and 25-fold to 100-fold reduction in infectivity compared with tRNA^{val} chimeras. The results indicated that the presence of the tRNA₃^{Lys}-rbz in 293 cells significantly reduced the titer of infectious virions produced from these cells. This demonstrates that the co-localization of the tRNA₃^{Lys}-rbz and the target HIV-1 DNA was mediated through specific binding of HIV RT to the tRNA₃^{Lys} moiety of the chimeric RNA.

d. Co-localization of Ribozyme and Target mRNA through the Binding of Each to Separate Units of a Protein Multimer, Such as Rev.

Ribozyme and target mRNA co-localization can be mediated through the binding of the ribozyme to one unit of a multimeric protein and the binding of the target mRNA to another unit of the multimer, thus bringing the target and ribozyme into close proximity.

This strategy is based upon an unsuccessful attempt by Konopka, et al. (56) to co-localize a ribozyme-aptamer with HIV-1 env mRNA through the binding of HIV-1 env Rev-responsive element (RRE) to Rev and the binding of the ribozyme-aptamer to a separate unit of the same Rev multimer. Rev is an early gene product of HIV which controls the expression of the HIV-1 structural genes through binding to a RRE and also facilitates the export of viral transcripts from the nucleus into the cytoplasm for translation of the viral structural proteins or assembly into viral structural particles. Rev occurs as a multimer. Interference with Rev-RRE interactions inhibits HIV replication. An RNA decoy, or aptamer, which acts by mimicking the major Rev binding site (RBE) within the RRE in the HIV-1 env mRNA has been shown to exert a significant inhibitory effect on HIV replication (57).

### EXAMPLE 4

Co-localization of ribozyme and target mRNA may be achieved by transfecting HeLa cells with plasmids in which an aptamer is combined with a ribozyme directed against the HIV-1 env mRNA. The target site of the anti-rev ribozyme is only 81 nucleotides upstream from the RRE in the viral mRNA. The aptamer-ribozyme and target HIV-1 mRNA may each bind to separate units of the Rev multimer thus bringing the ribozyme into proximity with its target. The aptamer-ribozyme should be more effective at inhibiting viral replication than the aptamer alone.

e. The 3′ Untranslated Region (UTR) as an RNA Trafficking Signal-Model for Ribozyme-Target Co-localization.

The factors which dictate the trafficking and intracellular localization of RNAs are poorly understood. There are some reports which suggest that RNAs may “track” along specific paths following transcription and transport to the cytoplasm (reviewed in 31). There are numerous examples of mRNAs which localize to specific regions of the cytoplasm as well. The most well studied localized RNAs are the oocyte and early embryo mRNAs of Drosophila and Xenopus (32). Other mRNAs such as actin have been shown to localize to cytoskeletal components (40, 41, 42). The signal for localization for many of the mRNAs which have been studied resides in the 3′ UTR (32, 42). Given that knowledge is limited as to how and why some mRNAs are localized to specific subregions of the cytoplasm, for the majority of targets it is difficult to design ribozymes which will be at the right place in the cell to maximize interactions with a given target RNA.

At this time, aside from the well-known actions of Rev on RRE containing transcripts, there is very little known about the role, if any, of the HIV UTR on intracellular partitioning of messenger RNAs. The nucleotide sequence of the region is uninformative, but the functions of the LTR, such as transcription termination and polyadenylation signaling, must be conserved. Since the 5′ and 3′ LTRs of retroviruses are identical, but have functionally different roles (transcription initiation for the 5′ LTR and termination and 3′ processing for the 3′ LTR), it is reasonable to ask whether placing a segment of the LTR at the 3′ end of a heterologous transcript will result in its functioning as a transcriptional termination polyadenylation signal. An intact HIV-1 LTR has been appended to the 3′ end of an insulin reporter gene and more than 98% of the transcripts were correctly processed and polyadenylated at the authentic poly A site (44, 45). It is therefore reasonable to test this region for its potential use as an mRNA localization signal. The following experiments are illustrative.

### EXAMPLE 5

Kislauskis, et al. (42) have demonstrated that the mRNAs encoding two actin isoforms, β-cytoplasmic and α-cardiac, can occupy different cytoplasmic compartments within the same cytoplasm of chicken fibroblasts. Moreover, the sequences in the respective actin 3′ UTRs were sufficient to localize a lacZ mRNA to the same cytoplasmic compartments. Actin isoforms contain very few differences in amino acid coding sequences, but the 3′ UTRs are isoform specific and evolutionarily conserved within a given isoform family, suggesting an important functional role (43).

These α- and β-actin UTRs can be used to co-localize ribozyme and target mRNAs intracellularly. Specifically, it has been shown that human α- and β-actin 3′ UTRs can be utilized as signals for co-localization of hammerhead ribozyme transcripts with a lacZ target mRNA substrate to the same subcellular compartments. The plasmid RSV β-gal, which contains a lacZ reporter gene, was used for constructing substrate RNA plasmids containing the human α- or β-actin 3′ UTRs. The human α-actin 3′ UTR was generated for human genomic DNA by PCR using the primers below.

Upper primer: 5′ AACTGCAGAT CTTCTAGACC CGGGCTAAGA TGCCTTCTCT CTCCATC 3′ (SEQ ID NO: 3)

Lower primer: 5′ GCTCTAGAAT TCGCTAGTCA CGTAACAATG CTCAGGGTGT CAAAGCA 3′ (SEQ ID NO: 4)

The 3′ UTR of β-actin was also derived by PCR from human genomic DNA.

Upper primer: 5′ AACTGCAGAT CTCTAGACCC GGGTAGGCGG ACTATGACTT AGTTGC 3′ (SEQ ID NO: 5)

Lower primer: 5′ AAGCTTGAAT TCGCTAGCTA CGTACCCACC CTCTGCTGCC CCCAACCA 3′ (SEQ ID NO: 6)

The hammerhead ribozyme motif used in this study contains 8 nucleotide binding arms complementary to the lacZ message indicated by underlined nucleotides in the ribozyme sequence below.

5′ UGUUUAUCCU GAUGAGUCCG UGAGGACGAA ACGGCUUA 3′ (SEQ ID NO: 7)

This was cloned into EcoRI-BamHI restriction sites of pBLUESCRIPT (pBSKS), yielding the BSKS Rbz plasmid, and used for in vitro transcription. For each transfection experiment, the lacZ HindIII-SnaI restriction fragments of each RSV β-gal 3′ UTR were replaced by the ribozyme containing HindIII-SnaI restriction fragments from pBSKS Rbz, yielding RSV Rbz 3′ α- or β-UTR. The HindIII-SnaI fragments containing both the ribozyme and each of the 3′ UTRs were cleaved from each RSV Rbz 3′ α- or β-UTR plasmid and inserted back into the HindIII-SnaI site of pHook2 (Invitrogen) (phook Rbz 3′ α- or β-UTR). In order to enzymatically follow the localization of the ribozymes, the humanized green fluorescent protein (GFP) was subcloned into the HindIII site of pHook2 Rbz 3′ UTR yielding pHook2 CMV-GFP-Rbz 3′ α- or β-UTRs.

To determine whether the 3′ UTRs of human actin mRNA could direct co-localization of ribozyme and substrate transcripts to the same subcellular compartments, pRSV-α-gal and pHook2 CMV-GFP-RBZ constructs fused to the α- or β-actin 3′ UTRs were cotransfected into chicken embryonic myoblasts and fibroblasts (CEMF). β-galactosidase activity measurements in cells co-transfected with substrate and pHook vector alone (without cloned ribozyme genes) were used as controls. 12-day-old CEMFs were transfected by the calcium phosphate coprecipitation procedure according to the supplier (CellPhect). The transient transfectants containing ribozyme genes cloned into the pHook2 vector were selected by a magnet according to the supplier (Invitrogen). For in situ hybridization, the selected transfectants were plated onto 0.5% gelatin-coated coverslips. The next day, cells were fixed in 4% formaldehyde and 10% glacial acetic acid in phosphate buffered saline and premeablized with 70% ethanol overnight at 4° C.

The distribution of RNAs was determined by using in situ hybridization performed using in vitro transcribed digoxigenin- or biotin-labeled probes to detect ribozyme or substrate RNAs, respectively. The RNA probes were constructed by linearizing pBSKS β-gal and pBSKS Rbz with HindIII and EcoRI, respectively, for T7 polymerase directed RNA synthesis. RNAs were labeled by incorporating either digoxigenin- or biotin-conjugated UTP according to the manufacturer's description (Boehringer Mannheim Biochem.) When the probes were more than 250 nt in length, mild alkaline hydrolysis was performed to generate smaller fragments of around 250 nt. The in situ hybridization was performed using these labeled probes. Cells were prehybridized with 2×SSC and 50% formaldehyde for 5 minutes at room temperature. Hybridization solutions were made fresh each time and contained 40 μg of denatured E. coli tRNA and 25-50 ng of labeled probe in 20 μl of 2×hybridization solution (4×SSC, 20% dextran sulfate, 0.02% BSA, 2 mM vanadyl ribonucleoside complex). The prehybridization solution was removed and 40 μl of fresh hybridization solution was added to each coverslip and incubated at 37° C. overnight in a humidified container. For soluble hybridizations, both probes were added simultaneously. Slides were washed twice in 0.1×SSC and 50% formaldehyde for 30 minutes at 50° C.

Immunostaining was used to detect ribozyme and substrate localization. For antibody staining, all reactions were performed for 30 minutes at 37° C. in 40 μl of antibody buffer (Ab:3×SSC and 10% formaldehyde) with 0.1% BSA and 2 mM vanadyl complex. After each reaction, the slides were washed twice with antibody for 15 minutes at room temperature. For detecting ribozyme localization, a 1/200 dilution of sheep anti-digoxigenin antibody (Boehringer Mannheim) followed by a 1/150 dilution of donkey anti-sheep antibody conjugated with FITC (Sigma) were utilized. For detecting substrate, a 1/2000 dilution of extravidin (Sigma), followed by a 1/2000 dilution of mouse anti-avidin conjugated with biotin (Sigma), followed by a 1/2000 dilution of extravidin conjugated with Cy3 (Sigma). Digital image processing was used to analyze localization of ribozyme and substrate RNAs within cells. The automated count function of Image-Pro Plus (Media Cybernetics) was used to count the number of yellow and blue signals for labeled cells and nucleus, respectively. Labeling intensity was analyzed after converting images to gray scale followed by pseudo coloring. The presence of co-localization is defined as the number of yellow cells images per number of blue cells images (total cells) in one image.

The immunostaining data from the probe hybridization studies revealed that the ribozyme-α-actin 3′ UTR transcripts preferentially localize to the perinuclear region (green, FITC) and the substrate containing the same 3′ UTR also preferentially localizes to this region (red, Cy3). Statistical analyses of the percent of transfectants showing overlap indicated that the matched 3′ UTR sequences significantly increased the frequency of co-localization of the ribozyme and its target (n=500). In cells containing matched α-actin 3′ UTRs (α-α), the percent of co-localization was increased 3.2-fold relative to unmatched 3′ UTRs (α-β) (22.9% versus 7.2%). Similarly, matched β-actin 3′ UTRs (β-β) increased the percent of co-localization by 2.8-fold relative to the non-homologous 3′ UTRs (β-α) (19.0% versus 6.7%). The data indicate that ribozymes and substrates containing matched 3′ UTRs show an approximate 3-fold increase in co-localization compared to constructs containing unmatched 3′ UTRs, and co-localization occurs in about 20% of the transfected CEMF cells using either the human α- or β-actin 3′ UTRs.

GFP, subcloned into the HindIII site of pHook2 Rbz 3′ UTR was used in order to enzymatically follow the localization of the ribozymes. GFP visualization was accomplished by incubating fixed cells in 0.2% X-gal solution followed by addition of 70% glycerol. The blue colored cells were identified under a phase-contrast light microscope. The distribution of GFP expression was utilized to indirectly visualize the subcellular localization of ribozyme and substrate mRNAs at their sites of translation.

Ribozyme-mediated inhibition of β-galactosidase activity was observed when matched 3′ UTRs were used. In order to examine whether or not this 3-fold increase in co-localization with matched 3′ UTRs translated into enhanced ribozyme function in transfected cells, β-galactosidase activity was assayed after magnetically selecting cells co-transfected width pHook-rbz and substrates containing matched and unmatched 3′ UTRs. Statistical analyses of the relative percent of β-galactosidase activity versus controls indicated that the most significant reduction in the β-galactosidase activity was achieved when ribozymes and lacZ target RNAs were co-localized with matched 3′ UTRs. Co-localization using the α-actin 3′ UTRs (α-α) resulted in 26% (63% versus 89%) and 6% (56% versus 62%) more reduction of β-galactosidase activity when using 1:1 and 1:5 ratios of substrate to ribozyme, respectively. The relative β-galactosidase activity of the matched (β-β) transcripts was reduced by 49% (21% versus 70%) compared to the unmatched localization signal (β-α) when the substrate to ribozyme ratio was 1:10. The reduction was 35% (54% versus 89%) at a 1:5 substrate to ribozyme ratio and only 4% (89% versus 93%) at a 1:1 ratio.

### EXAMPLE 6

A similar approach to utilizing 3′ UTRs for co-localization of target and inhibitor involves using the HIV-1 3′ UTR, which is present in all HIV transcripts. The basic strategy is to incorporate the 3′ UTR of interest onto a reporter construct as well as to incorporate the same UTR onto a ribozyme transcript. The 293 and HeLa cell lines were used for the studies. The reporter construct to be used is depicted below and contains the human growth hormone (GH) gene driven by the SIV-1 LTR promoter. This system produces a readily quantifiable (using a radioimmunoassay) secreted protein. The linear range of response of GH expression to plasmid concentration in the 293 cell line was established. The expression of this construct is not dependent upon TAT expression, although a 10 fold stimulation of expression in the presence of SIV TAT was observed. If the results look promising in the 293 cell line, confirmation testing in HeLa cells will be carried out. The 3′ UTRs will be appended to both the growth hormone and ribozyme expression cassettes. To do this, the human β-actin or α-actin 3′ UTRs will be isolated from human genomic DNA or mRNAs utilizing PCR. The primers for isolating the two human actin 3′ UTRs are:

beta actin oligo 5′

5′AGATCTTCTAGACCCGGGTAGGCGGACTATGACTTAGTTGC3′ (SEQ ID NO. 9)

beta actin oligo 3′

5′GAATTCGCTAGCTACGTACCCACCCTCTGCTGCCCCCAAC3′ (SEQ ID NO. 10)

alpha actin oligo 5′

5′AGATCTTCTAGACCCGGGCTAAGATGCCTTCTCTCTCCATC3′ (SEQ ID NO. 11)

alpha actin oligo 3′

5′GAATTCGTCAGCTACGTAACAATGCTCAGGGTGTCAAAGCA3′ (SEQ ID NO. 12)

The ribozyme will be expressed utilizing the RSV promoter with the appropriate actin UTR appended to the 3′ end. Utilizing transient transfection of the reporter constructs into pools of stably transfected ribozyme containing cells, the effect of the ribozyme mediated inhibition of the reporter construct will be monitored. Ribozyme constructs may be made in the adeno associated virus vector backbone. The constructs will be encapsidated, and transduced into three A293 or HeLa cell lines. Stable lines will be selected from G418, and levels of ribozyme expression will be monitored via primer extension and northern gel analyses. For each ribozyme, a non-cleaving mutant control will be used. The controls for 3′ UTR effects will utilize comparison of the efficiency of reporter gene inhibition as a function of having the β- versus α-actin 3′ UTRs, which localize to different intracellular compartments, appended to the reporter and ribozyme transcripts. Several ribozyme targets in the SIV leader region have been established which will be tested in conjunction with the UTRs. These ribozymes have been tested for substrate interaction using an in vitro gel shift assay, and identified by this process sites in the SIV LTR which are most accessible to binding. In each case where binding was shown to be efficient, good cleavage activity by the ribozyme was observed.

The first set of sequences appended to the GH reporter construct included the last 20 bases of the pNL4-3 proviral nef coding sequence and extended to the 3′ terminus of the LTR. Much of this region is included in all of the viral messenger and full length genomic transcripts. This sequence contains the poly A additional signal and putative transcriptional termination region (45), but most importantly lacks cis-acting regulatory signals such as the RRE, INS and CRS. This region was isolated using PCR primers and appended to both the GH reporter gene construct and the ribozyme transcriptional units as described above.

The control constructs included the AAV poly A and termination signals, which were appended to the ribozyme and GH reporter constructs as well as mutant, non-cleaving ribozymes. Again, efficacy was measured by inhibition of GH secretion in transient transfection assays of the GH construct into stable cell lines expressing the ribozyme constructs as described above.

f. Co-Localization of Anti-HIV-1 Ribozymes with Full Length Viral Transcripts Via the Dimerization Domain and the Viral RRE.

All retroviruses require packaging of two genomic RNAs for infectivity. The viral sequences that direct packaging are known as the psi (ψ) element. Retroviral genomic RNAs are linked close to their 5′ ends in a parallel orientation forming dimers, and the dimerized region maps within the ψ element. Consequently, dimerization is thought to be required in vivo both in selective encapsidation of unspoiled viral genomic-length RNAs and in the early steps of replication occurring immediately postinfection.

The dimertization or packaging strategy for co-localizing ribozyme and target RNAs will capitalize upon the unique RNA-RNA interaction of the dimerization domain of HIV (which is facilitated by the NCp7 nucleocapsid protein) (46-49) in combination with the RRE to force cytoplasmic translocation of the ribozyme containing transcripts. The rationale for these studies is that ribozyme containing RNAs which harbor the signals required for packaging can be co-localized with unspliced viral mRNAs and genomic RNAs via interactions of the dimerization domains. The most probable targets for ribozyme interactions will be full-length viral RNAs, destined for encapsidation or translation into viral structural proteins.

These experiments are based upon the success of a somewhat similar strategy employed by Sullenger and Cech (33). Sullenger and Cech demonstrated ψ-dependent co-localization of a retroviral vector RNA expressing a ribozyme with a ψ-tethered target. Their results demonstrated that copackaging of these two RNAs into virions resulted in reduction of the titer of lacZ transduction by 90%, clearly a function of ribozyme destruction of the target RNA. However, Sullenger and Cech demonstrated co-localization of ribozyme and target mRNA only after virion packaging and not in the cell cytoplasm as evidenced by the absence of ribozyme-mediated activity in the cytoplasm and its presence after virion packaging and transduction of target cells.

### EXAMPLE 7

The approach of Sullenger and Cech was modified to allow ribozymes tethered to ψ elements (which contain the dimerization region) and their targets to co-localize in the cytoplasm of both packaging and non-packaging cells (58). For these experiments, three Retroviral vector constructs were made. The first, pψneo was derived from the MLV vector pLNL-6 which utilize antibiotic resistance markers to monitor transfection efficiency. All of the constructs are deficient in the coding regions for pol, env, and the major part of the gag gene but retain the sequences known to be required for dimerization and packaging (the ψ region). The second construct, pψhygRbz, has the neo gene replaced by the hygro gene, includes the MLV ψ sequences, and has a functional anti-neo ribozyme appended immediately after the hygro coding region. The third construct, pψhyg*Rbz, is the same as pψhygRbz except the ribozyme is crippled by a double point mutation.

The ribozyme and target retroviral vectors were transfected and/or cotransfected into the murine Retroviral PA317 (American Type Culture Collection) packaging cell line to take advantage of constitutively produced viral proteins that enhance the dimerization of the viral RNAs in vitro. The PA317 cell line carries truncated mouse retroviral DNA (lacking the ψ element, the 3′ LTR, and part of the 5′ LTR) and the herpes simplex thymidine kinase gene (both in pBR322). Cells were transfected according to the manufacturer's protocol (CellPhect) using the following plasmid combinations: (1) pψneo alone, (2) pψneo plus pψhygRbz, and (3) pψneo plus pψhyg*Rbz (2:1 ratio of Rbz to target vectors; 9 μg of total plasmid DNA). Selection was performed with 500 μg/ml G418 (Mediatech) and 50 μg/ml hygromycin (Sigma). The number of stably transfected G418-resistent colonies was reduced approximately 100-fold in the presence of the functional ribozyme and 2-fold in the presence of the crippled ribozyme relative to colonies arising after transfection with pψneo alone. These results are consistent with a specific ribozyme effect.

In order to verify that the reduction in G418resistant clones was a consequence of ribozyme-mediated destruction of the neo transcript and was not due to nonspecific toxicities, an experiment was performed using the same constructs but scoring separately for G418 and hygromycin resistance. To assay for co-localization, the cells were divided equally after transfection and one half was subjected to G418 and the other half to hygromycin B (50 μg/ml) selection. Since only the ribozyme and mutant ribozyme construct carry the hygro gene, the number of hygromycin-resistant colonies serves to normalize the transfection efficiencies. The number of G418-resistant colonies monitored neo gene expression from the target vectors. As observed in the first set of experiments, the functional ribozyme severely inhibited the formation of G418-resistant colonies, whereas the mutant ribozyme had little effect.

In order to evaluate the role of nucleocapsid proteins in dimerization as measured by ribozyme-mediated inactivation of the neo transcript in vivo, the transfection assay was repeated using the human 293 nonpackaging cell line. The protocol was identical to that used with PA317 cells. The results obtained from these transfection experiments suggest that intracellular dimerization and consequent ribozyme-target co-localization can occur in the absence of the viral nucleocapsid proteins, albeit less efficiently than in their presence. The data from the 293 cell transfections confirm that the presence of the ψ element is essential for this process.

Sullenger and Cech demonstrated ψ-dependant co-localization of a retroviral vector RNA expressing a ribozyme with a ψ-tethered lacZ target. Their results demonstrated that copackaging of these two RNAs into virions resulted in reduction of the titer of lacZ transduction by 90%, clearly a function of ribozyme destruction of the target RNA. They were unable to detect ribozyme-medicated reduction of LacZ activity in the cell cytoplasm and saw the ribozyme effect only after virion packaging and transduction of target cells. A difference between their experimental approach and the present invention is that their target RNA was expressed from a stably transformed cell line and the ribozyme-encoding viral vector was transduced into these cells. In contrast, this approach co-transfects the two DNAs into the same cells, thereby allowing coordinated expression of the RNAs and increasing the likelihood that the ψ elements from the two vectors could interact. In addition, the data herein show that >90% of the transcripts generated by pψneo and derivatives containing other ribozymes are unspliced and therefore contain the entire ψ domain, which optimizes chances for co-localization in these experiments. Additionally, the results presented here demonstrate that ψ-mediated ribozyme-target heterodimerization can occur independently of packaging.

### General Purpose or Utility of the Invention

Co-localization mechanisms and the resulting living cells which include co-localized inhibitors and targets are disclosed. HIV and other lentiviral RNAs co-localized with a ribozyme provide intracellular and therapeutic agents and vaccines for mammalian lentiviral infections. Such therapeutic agents and vaccines are administered in known manner by viral mediated delivery, e.g., AAV or retroviral deliveries.

The idea of chimeric tRNA^{Lys}-ribozyme molecules which effectively compete with tRNA^{Lys} for binding to HIV-1 RT is novel. It provides a possible mechanism for specifically delivering inhibitors of HIV-1 RT to the virion particle itself. Such inhibitory agents will render these viral particles non-functional, and thus serve as highly specific, nontoxic therapeutic agents.

It has been demonstrated that the entire tRNA^{Lys} molecule, as well as various segments of the tRNA itself, are capable of specifically interacting with HIV-1 RT. No one has shown that chimeric molecules such as the ones described could specifically bind to HIV-1 RT thereby affecting polymerase activity. There is one published report of an RNAse cleavage activity associated with HIV-1 RT. This activity was only shown to cleave HIV-1 RNA, not the primer. This activity cleaves twice in the PBS, and only substrates paired with tRNA^{Lys.}

The RNA attached at the 3′ end of the tRNA^{Lys} need not be a ribozyme, but any extra RNA which can base pair with the HIV-1 target upstream of the primer binding site. If a ribozyme is joined to the tRNA, other cleavage sites such as CUC, or CUA which are on the HIV-1 sequence just to the 3′ side (downstream) of the AUC site, can be targeted. It is not necessary to make an entire tRNA^{Lys}-ribozyme fusion because it is now known that the last 18 nucleotides of tRNA^{Lys} fused to the ribozyme are also bound by HIV-1 RT. Genetic variants of tRNA^{Lys} which compete better than tRNA^{Lys} for binding to HIV-1 transcriptase are included in the invention.

The ribozyme fusions to tRNA^{Lys} allow specific targeting of the ribozyme to HIV-1 virion. Since all retroviruses use cellular tRNAs for priming, this invention provides a general strategy for inhibiting other retroviruses as well. Existing ribozyme technology makes use of specific base pairing between ribozyme and target, but this is accomplished by diffusion of the ribozyme until it finds a target RNA. This invention uses well-known retroviral packaging pathways to specifically carry the ribozyme into the virion, and get it bound to the correct site on the viral RNA for cleavage.

### REFERENCES CITED

1. Kruger, K., et al., Cell 31:147-157 (1982).

2. Guerrier-Takada, C., et al., Cell 35:849-857 (1983).

3. Cech, T. R., Current Opinion in Structural Biology 2:605-609 (1992).

4. Rossi, J. J., Biotechnology 3:3-7 (1992).

5. Castanotto, D., et al., Critical Reviews in Eukaryotic Gene Expression 2:331-357 (1992).

6. Symons, R. H., Ann. Rev. Biochem. 61:641-671 (1992).

7. von Ahsen, U., et al., BioEssays 15:299-307 (1993).

8. Dahm, S. C., et al., Biochemistry 32:13040-13045 (1993).

9. Taira, K., et al., Protein Engineering 3:691-701 (lsso).

10. Uchimaru, T., et al., The FASEB Journal 7:137-142 (1993).

11. Chang, P., et al., Clinical Biotech 2:23-31 (1990).

12. Sarver, N., et al., Science 247:1222-1225 (1990).

13. Larder, B. A., et al., Science 243:1731-1734 (1989).

14. Chen, C. J., Nucleic Acids Research 20:4581-4589 (1992).

15. Vaishav, et al., Ann. Rev. Biochem. 60:577-630 (1991).

16. Yu, M., et al., Gene Therapy 1:13-26 (1994).

17. Zack, J. A., et al., Cell 61:213-222 (1990).

18. Yamada, O., et al., Gene Therapy 1:38-45 (1994).

19. Crisell, P., et al., Nucleic Acids Research 21:52515255 (1993).

20. Dropulic, B., et al., Journal of Virology 66:14321441 (1992).

21. Lo, K. M. S., et al., Virology 190:176-183 (1992).

22. Ojwang, J. O., et al., Proc. Natl. Acad. Sci. USA 89:10802-10806 (1992).

23. Weerasinghe, M., et al., Journal of Virology 65:5531-5534 (1991).

24. Yu, M., et al., Proc. Natl. Acad. Sci. USA 90:63406344 (1993).

25. Fauci, A. S., et al., Science 262:1011-1018 (1993).

26. Pantaleo, G., et al., Nature 362:355-358 (1993).

27. Embretson, J., et al., Massive covert infection of helper t. lymphocytes and macrophages by HIV during the incubation period of AIDS, 362:359-362 (1993).

28. Goodenow, M., et al., J. AIDS 2:344-352 (1989).

29. Canoga, Y., et al., Science 236:819-822 (1987).

30. Looney, D. J., et al., Science 241:357-359 (1988).

31. Rosbash, M., et al., Cell 75:399-401 (1993).

32. Ding, D., et al., BioEssays 15:651-658 (1993).

33. Sullenger, B. A., et al., Science 262:1566-1569 (1993).

34. Citovsky, V., et al., Ann. Rev. Microbiol. 47:167197 (1993).

35. Tsuchihashi, Z., et al., Science 267:99-102 (1993).

36. Herschiag, D., et al., The EMBO Journal 13:2913-2924 (1994).

37. Bertrand, E., et al., The EMBO Journal 13:2904-2912 (1994).

38. Litvak, S., et al., TIBS 19:114-118 (1994).

39. Parry, H., et al., TIBS 14:15-19 (1989).

40. Bentley, L., et al., Cell 45:407-415 (1986).

41. Cheng, H., et al., J. Mol. Biol. 210:541-549 (1989).

42. Kislauskas, E. H., et al., The J. of Cell Biology 123:165˜172 (1993).

43. Yaffe, D., et al., Nucl. Acids Res. 13:3723-3737 (1985).

44. Bohnlein, S., et al., J. Virol. 63:421-424 (1989).

45. Guntaka, R. V., Microbiological Reviews 57:511-521 (1993).

46. Awang, G., et al., Biochemistry 32:11453-11457 (1993).

47. Darlix, J. L., et al., J. Mol. Biol. 216:689-699 (1990).

48. Gorelick, R. J., et al., J. Virol. 64:3207-3211 (1990).

49. DeRocquigny, H., et al., Proc. Natl. Acad. Sci. USA 89:6472-6476 (1992).

50. Gene 149:33-39 (1994).

51. Barat, et al. EMBO Journal 8:3279-3285 (1989).

52. Khan, et al. J. Bio. Chem 267:6689-6695 (1992).

53. Weiss, et al., Gene 111:183-197 (1992).

54. Ben-Artzi, Proc. Natl. Acad. Sci. USA 89:927-931 (1992).

55. Westaway, et al., Antisense & Nucleic Acid Drug Devel. 8:185-197 (1998).

56. Konopka, K. et al., J. Drug Targeting 5: 247-259 (1998).

57. Lee, et al., J. Virol. 68: 8254-8264 (1994).

58. Pal, et al., J. Virol. 72:8349-8353 (1998).

What is claimed is:

1. A process which comprises the positioning, within a living cell, of a target molecule and an inhibitor for said target molecule, said positioning being such that the concentration of the inhibitor molecule with respect to the target molecule is enhanced.

2. The claim 1 process in which the target molecule is an RNA molecule and the inhibitor is a ribozyme.

3. The claim 1 process in which the target molecule is an HIV-1 RNA molecule and the inhibitor is a ribozyme which cleaves said HIV-1 RNA molecule.

4. A method which comprises co-localizing a target molecule and an inhibitor for said target molecule within a living cell.

5. A living cell in which a target molecule and an inhibitor for said target molecule are co-localized.

6. The claim 4 method in which the target molecule is an RNA molecule and the inhibitor is a ribozyme which cleaves said RNA molecule.

7. The living cell of claim 5 in which the target molecule is an RNA molecule and the inhibitor is a ribozyme which cleaves said RNA molecule.

8. A method which comprises co-localizing within a living mammalian cell
an RNA target molecule, and
a ribozyme which cleaves said RNA target molecule said ribozyme including
(i) the dimerization or packaging signal of said RNA target molecule, or
(ii) a sequence capable of pairing with said RNA target molecule at a site upstream of a tRNA₃^{Lys} binding site on said RNA target molecule wherein said ribozyme is bound to a tRNA₃^{Lys} molecule at the 3′ end of said tRNA₃^{Lys}, or
(iii) a 3′ untranslated region (UTR) of said RNA molecule, or
(iv) a sequence capable of binding to a cellular protein to which the target RNA also binds, or
(v) a sequence capable of binding to a unit of a multimeric cellular protein such that the target RNA binds to the same or another unit of the multimer.

9. The claim 8 method in which said RNA target molecule is an HIV-I RNA molecule.
